(19) United States
(12) Patent Application Publication
(10) Pub. No.: US 2007/0225500 A1
(43) Pub. Date: Sep. 27, 2007

(54) Process for the Preparation of Pyridin-2-Ylmethylsulphinyl-1H-Benzimidazol Compounds

(75) Inventors: Bernhard Kohl, Konstanz (DE); Bernd Mueller, Konstanz (DE); Ralf Steffen Weingart, Konstanz (DE)

Correspondence Address: NATH & ASSOCIATES PLLC, 112 South West Street, Alexandria, VA, 22314, US

(73) Assignee: Altana Pharma AG, Konstanz (DE)

(21) Appl. No.: 11/597,373

(22) PCT Filed: May 31, 2005
(86) PCT No.: PCT/EP05/52471

§ 371 Date: Nov. 22, 2006

(30) Foreign Application Priority Data
Jun. 2, 2004 (EP) 04102467.0

(51) Int. Cl. C07D 401/12

(52) U.S. Cl. 546/268.1

(57) ABSTRACT

The invention relates to a novel process for preparing mixtures of enantiomers of PPI's having a sulphinyl structure using a mixture of enantiomers of chiral zirconium complexes or chiral hafnium complexes.

### SUBJECT-MATTER OF THE INVENTION

The present invention relates to a novel process for preparing pure PPI's which can be used for preparing medicaments in the pharmaceutical industry.

### TECHNICAL BACKGROUND

Pyridin-2-ylmethylsulphinyl-1H-benzimidazoles and compounds of a closely related structure, as known, for example, from EP-A-0005129, EP-A-0166287, EP-A-0174726 and EP-A-0268956, are, owing to their H⁺/K⁺-ATPase-inhibitory action, of considerable importance in the therapy of diseases associated with an increased secretion of gastric acid.

Examples of active compounds from this class of compounds which are commercially available or in clinical development are 5-methoxy-2-[(4-methoxy-3,5-dimethyl-2-pyridinyl)methylsulphinyl]-1H-benzimidazole (INN: omeprazole), (S)-5-methoxy-2-[(4-methoxy-3,5-dimethyl-2-pyridinyl)methyl-sulphinyl]-1H-benzimidazole (INN: esomeprazole), 5-difluoromethoxy-2-[(3,4-dimethoxy-2-pyridinyl)methylsulphinyl]-1H-benzimidazole (INN: pantoprazole), 2-[3-methyl-4-(2,2,2-trifluoroethoxy)-2-pyridinyl)methylsulphinyl]-1H-benzimidazole (INN: lansoprazole), 2-{[4-(3-methoxypropoxy)-3-methylpyridin-2-yl]methylsulphinyl}-1H-benzimidazole (INN: rabeprazole) and 5-methoxy-2-((4-methoxy-3,5-dimethyl-2-pyridylmethyl)sulphinyl)-1H-imidazo(4,5-b)pyridine (INN: tenatoprazole).

The abovementoned sulphinyl derivatives which, owing to their mechanism of action, are also referred to as proton pump inhibitors or abbreviated PPI are chiral compounds. The process usually used for preparing the PPI is the oxidation of the corresponding sulphides.

### PRIOR ART

The international patent application WO99/25711 (which corresponds to U.S. Pat. No. 6,303,788) describes a process for the preparation of omeprazole by oxidation of the corresponding sulphide in the presence of a titanium complex and optionally in the presence of a base.

The international patent application WO96/02535 (which corresponds to U.S. Pat. No. 5,948,789) describes a process for the enantioselective synthesis of PPI using chiral titanium complexes. What is described is, inter alia, the synthesis of (+)- and (−)-[or, expressed in a different way, (R)- and (S)]-pantoprazole, the chiral auxiliary used for the synthesis of (+)-pantoprazole being diethyl (+)-tartrate and the chiral auxiliary used for the preparation of (−)-pantoprazole being diethyl (−)-tartrate.

U.S. Pat. No. 3,449,439 describes a process for the production of organic sulfones from organic sulfides or organic sulfoxides by reacting the starting compound with an organic hydroperoxide in the presence of a catalyst selected from compounds of titanium, molybdenum and vanadium.

The enantioselective sulphoxidation for preparing esomeprazole ((S)-omeprazole) on a large scale using a chiral titanium complex is described in Tetrahedron, Asymmetry, (2000), 11, 3819-3825.

The enantioselective sulphoxidation of aryl alkyl sulphides and dialkyl sulphides in the presence of a zirconium catalyst having a polydentate ligand is described in J. Org. Chem., (1999), 64(4), 1327.

### DESCRIPTION OF THE INVENTION

The invention provides a process for preparing mixtures of enantiomers of PPI's having a sulphinyl structure. The process is characterized in that the oxidation of the corresponding sulphide is carried out in the presence of a mixture of enantiomers of chiral zirconium complexes or chiral hafnium complexes and in the presence of a mixture of enantiomers of D/L-tartaric acid derivatives.

A preferred embodiment of the invention is a process for preparing racemic mixtures of PPI's having a sulphinyl structure. The process is characterized in that the oxidation of the corresponding sulphide is carried out in the presence of a racemic mixture chiral zirconium complexes or chiral hafnium complexes and in the presence of a racemic mixture of D/L-tartaric acid derivatives.

The oxidation is advantageously carried out in an organic solvent, such as, for example, ethyl acetate, toluene, dichloromethane, dioxane or, preferably, methyl isobutyl ketone, where it is not necessary for the solvents mentioned to be completely anhydrous or where anhydrous solvents are in each case optionally admixed with a defined proportion of water, for example up to a maximum of 0.5 equivalent. For reactions with less than 0.5 equivalent of zirconium or hafnium complex, it is preferred to use an anhydrous solvent. The solvents employed may be used in the commercially available quality.

A solvent essentially comprises a specific solvent if it contains at least 50%, preferably at least 90%, in particular at least 95%, of said specific solvent. An anhydrous solvent is essentially free of water, having a water content of less than 5%, preferably less than 1%, in particular less than 0.3%.

Suitable oxidizing agents are all anhydrous oxidizing agents customarily used for the synthesis of PPI, where particular mention may be made of hydroperoxides, such as, for example, tert-butyl hydroperoxide or, in particular, cumene hydroperoxide. In general, 0.90 to 1.3 oxidation equivalents, preferably 0.95-1.05 equivalents, of the oxidizing agent are used.

The zirconium or hafnium complex suitable for catalyzing the process of the present invention is prepared from a mixture of enantiomers of D/L-tartaric acid derivatives and a zirconium or hafnium (IV) component.

Suitable zirconium components are, for example, zirconium(IV) acetylacetonate, zirconium(IV) butoxide, zirconium(IV) tert-butoxide, zirconium(IV) ethoxide and, in particular, zirconium(IV) n-propoxide (preferably as a solution in n-propanol) or zirconium(IV) isopropoxide (preferably in the form of the zirconium(IV) isopropoxide/isopropanol complex). Suitable hafnium components are, for example, hafnium(lV) acetylacetonate, hafnium(IV) butoxide, hafnium(IV) n-propoxide, hafnium(IV) isopropoxide (preferably in the form of the hafnium(IV) isopropoxide/isopropanol complex), hafnium(IV) ethoxide and in particular hafnium(lV) tert-butoxide. Preference is given to using a zirconium component.

In general, 0.01-2 equivalents, preferably 0.05-0.9 equivalent, of the zirconinum component or of the hafnium component are used.

Suitable mixtures of enantiomers of D/L-tartaric acid derivatives are, for example D/L-tartaric acid amides, such as D/L-tartaric acid bis-(N,N-diallylamide), D/L-tartaric acid bis-(N,N-dibenzylamide), D/L-tartaric acid bis-(N, N-diisopropylamide), D/L-tartaric acid bis-(N,N-dimethylamide), D/L-tartaric acid bis-(N-pyrrolidinamide), D/L-tartaric acid bis-(N-piperidinamide), DIL-tartaric acid bis-(N-morpholinamide), D/L-tartaric acid bis-(N-cycloheptylamide) or D/L-tartaric acid bis-(N-4-methyl-N-piperazinamide), or dialkyl D/L-tartrates, such as dibutyl D/L-tartrate, di-tert-butyl D/L-tartrate, diusopropyl D/L-tartrate, dimethyl D/L-tartrate and diethyl D/L-tartrate.

The mixture of enantiomers of D/L-tartaric acid derivatives comprises mixtures of the D-tartaric acid erivative and the L-tartaric acid derivatives in any mixing ratio. Preferably, the mixture of enantiomers of D/L-tartaric acid derivatives is a racemic mixture comprising equal amounts of the D-tartaric acid derivative and the L-tartaric acid derivative. The use of a racemic mixture of D/L-tartaric acid derivatives in the process according to the invention leads to a racemic mixture of the PPI.

In general, 0.02-4 equivalents, preferably 0.1-2 equivalents, of the mixture of enantiomers of D/L-tartaric acid derivatives is employed.

Particularly preferred mixtures of enantiomers of D/L-tartaric acid derivatives are D/L-tartaric acid bis-(N,N-dimethylamide), D/L-tartaric acid bis-(N-pyrrolidinamide) and D/L-tartaric acid bis-(N-morpholinamide).

A mixture of enantiomers of D/L-tartaric acid derivatives to be emphasized is D/L-tartaric acid bis-(N-pyrrolidinamide).

Particularly suitable for the preparation of a mixture of enantiomers of pantoprazole are mixtures of enantiomers of D/L-tartaric acid bis-(N,N-dimethylamide), D/L-tartaric acid bis-(N-pyrrolidinamide) and D/L-tartaric acid bis-(N-morpholinamide).

For the preparation of a mixture of enantiomers of pantoprazole the use of a mixture of enantiomers of D/L-tartaric acid bis-(N-pyrrolidinamide) is emphasized.

The oxidation is preferably carried out at temperatures between −20 and 50° C., in particular at room temperature and optionally in the presence of a base, suitable bases being, in particular, organic bases, preferably a tertiary amine, such as triethylamine or N-ethyldiisopropylamine.

If the process is carried out in a suitable manner, the pure PPI having sulphinyl structure is obtained a purity of >98%. By further steps, such as, for example, pH-controlled reprecipitation and/or recrystallization in a suitable solvent, such as, for example acetonitrite or isopropanol, it is possible to further increase the purity considerably. Reprecipitation is carried out via intermediate preparation of suitable salts, such as, for example, via the sodium salt (for other possible salts, see, for example, EP-A-166287).

The invention is illustrated in more detail by the examples below, but not limited in any way. The abbreviation h stands for hour(s).

### EXAMPLES

### 1. 5-Difluoromethoxy-2-[(3,4-dimethoxy-2-pyridinyl)methylsulphinyl]-1H-benzimidazole with a mixture of D- and L-tartaric acid bis-(N,N-pyrrolidinamide) and zirconium(IV) n-propoxide

At room temperature, 50.0 g of 5-difluoromethoxy-2-[(3,4-dimethoxy-2-pyridinyl)methylthio]-1H-benzimidazole were suspended in 100 ml of methyl isobutyl ketone together with 7.0 g of racemic D/L-tartaric acid bis-(N-pyrrolidinamide) and 6.1 ml of zirconium(IV) n-propoxide (70% in propanol). The mixture was heated at 40° C. for one hour, resulting in the formation of a solution which is almost clear. After cooling to room temperature, 1.8 ml of N-ethyidiisopropylamine were added and 26.9 ml of cumene hydroperoxide were then slowly metered in. Stirring was continued at room temperature until the oxidation has ended (5-24 hours, monitored by TLC). The clear solution was diluted with 100 ml of methyl isobutyl ketone and quenched with 1.7 g of sodium thiosulphate in 200 ml of saturated sodium bicarbonate solution and stirred for a further 14 hours. 120 ml of isopropanol were added and after phase separation, the mixture was washed twice with 100 ml of saturated sodium bicarbonate solution. 350 ml of water were added to the methyl isobutyl ketone phase, and the pH was adjusted to pH=13 using a 40% by weight strength aqueous solution of sodium hydroxide. After phase separation, the methyl isobutyl ketone phase was extracted with another 100 ml of water at pH=13. The aqueous phases were combined and, at 40° C., subjected to incipient distillation under reduced pressure and filtered over Hyflo. Pantoprazole was precipitated by addition of 10% strength acetic acid to pH=9.0. Stirring was continued for another 12 hours during which the pH was monitored. The beige crystals were filtered off and washed with 100 ml of water. The title compound was obtained in a yield of about 16 g (75% of theory)

To increase the purity, Pantoprazole was dissolved in water/aqueous sodium hydroxide solution at pH=13 and re-precipitated with acetic acid (10%) at pH=9.0. This step was repeated one time. Pantoprazole, the title compound was isolated as almost colorless crystals.

yield: 27 g (52% of the theory) m.p.: 137-138° C. (decomposition) Chemical purity: >98% area percent (HPLC) Optical rotation: α²⁰_{D}=0° (c=0,5 MeOH)

### 2. 5-Difluoromethoxy-2-[(3,4-dimethoxy-2-pyridinyl)methylsulphinyl]-1H-benzimidazole

Analogously to Example 1, reaction of 5-difluoromethoxy-2-[(3,4-dimethoxy-2-pyridinyl)methylthio]-1H-benzimidazole under otherwise identical conditions, but without addition of N-ethyidiisopropylamine, gave the title compound in a yield of 50% of theory and a purity of >98%.

### 3. 5-Difluoromethoxy-2-[(3,4-dimethoxy-2-pyridinyl)methylsulphinyl]-1H-benzimidazole with catalytic amounts of D/L-tartaric acid bis-(N-Pyrrolidinamide) and zirconium(IV) iso-propoxide

Analogously to Example 1, reaction of 5-difluoromethoxy-2-[(3,4-dimethoxy-2-pyridinyl)methylthio]-1H-benzimidazole under otherwise identical conditions, but with 0.1 equivalent of zirconium iso-propoxide, 0.125 equivalents of racemic D/L-tartaric acid bis-(N-pyrrolidinamide) and 0.07 equivalents of triethylamin gave, after an oxidation time of 48-72 h, the title compound in a yield of 50% of theory and a purity of >98%.

1. A process for preparing a mixture of enantiomers of a PPI having a sulphinyl structure comprising oxidizing a corresponding sulphide in the presence of a mixture of enantiomers of chiral zirconium complexes or chiral hafnium complexes.

2. A process for preparing a mixture of enantiomers of a PPI having a sulphinyl structure comprising oxidizing a corresponding sulphide in the presence of a mixture of enantiomers of chiral zirconium complexes.

3. The process according to claim 1, characterized in that the oxidation is carried out using cumene hydroperoxide.

4. The process according to claim 1, characterized in that zirconium(IV) acetylacetonate, zirconium(IV) butoxide, zirconium(IV) tert-butoxide, zirconium(IV) ethoxide, zirconium(IV) n-propoxide, zirconium(IV) isopropoxide or zirconium(IV) isopropoxide/isopropanol complex or hafnium(IV) acetylacetonate, hafnium(IV) butoxide, hafnium(IV) tert-butoxide, hafnium(IV) ethoxide, hafnium(IV) n-propoxide, hafnium(IV) isopropoxide or hafnium(IV) isopropoxide/isopropanol complex is used.

5. The process according to claim 2, characterized in that zirconium(IV) acetylacetonate, zirconium(IV) butoxide, zirconium(IV) tert-butoxide, zirconium(IV) ethoxide, zirconium(IV) n-propoxide, zirconium(IV) isopropoxide or zirconium(IV) isopropoxide/isopropanol complex is used.

6. The process according to claim 1, characterized in that the process is carried out in the presence of a mixture of enantiomers of D/L-tartaric acid derivatives.

7. The process according to claim 1, characterized in that the process is carried out in the presence of a racemic mixture of D/L-tartaric acid derivatives.

8. The process according to claim 1, characterized in that the process is carried out in the presence of a mixture of enantiomers of D/L-tartaric acid bis-(N,N-diallylamide), D/L-tartaric acid bis-(N,N-dibenzylamide), D/L-tartaric acid bis-(N,N-diisopropylamide), D/L-tartaric acid bis-(N,N-dimethylamide), D/L-tartaric acid bis-(N-pyrrolidinamide, D/L-tartaric acid bis-(N-piperidinamide), D/L-tartaric acid bis-(N-morpholinamide), D/L-tartaric acid bis-(N-cycloheptylamide), D/L-tartaric acid bis-(N-4-methyl-N-piperazinamide), dibutyl D/L-tartrate, di-tert-butyl D/L-tartrate, diisopropyl D/L-tartrate, dimethyl D/L-tartrate or diethyl D/L-tartrate.

9. The process according to claim 1, characterized in that the process is carried out in the presence of a mixture of enantiomers of D/L-tartaric acid bis-(N,N-dimethylamide), D/L-tartaric acid bis-(N-pyrrolidinamide) or D/L-tartaric acid bis-(N-morpholinamide).

10. The process according to claim 1, characterized in that the oxidation is carried out in the presence of an organic base.

11. The process according to claim 1, characterized in that the oxidation is carried out in the presence of a tertiary amine.

12. The process according to claim 1, characterized in that the oxidation is carried out in organic solvents.

13. The process according to claim 1, characterized in that the oxidation is carried out in organic solvents comprising 0 to 0.3% by volume of water.

14. The process according to claim 1, characterized in that the oxidation is carried out in an organic solvent which comprises methyl isobutyl ketone.

15. The process according to claim 1, characterized in that the zirconium component used is zirconium(IV) acetylacetonate, zirconium(IV) butoxide, zirconium(IV) tert-butoxide, zirconium(IV) ethoxide, zirconium(IV) n-propoxide, zirconium(IV) isopropoxide, or zirconium(IV) isopropoxide/isopropanol complex, and that the process is carried out in the presence of a mixture of enantiomers of D/L-tartaric acid bis-(N,N-diallylamide), D/L-tartaric acid bis-(N,N-dibenzylamide), D/L-tartaric acid bis-(N,N-diisopropylamide), D/L-tartaric acid bis-(N,N-dimethylamide), D/L-tartaric acid bis-(N-pyrrolidinamide), D/L-tartaric acid bis-(N-piperidinamide), D/L-tartaric acid bis-(N-morpholinamide), D/L-tartaric acid bis-(N-cycloheptylamide), D/L-tartaric acid bis-(N-4-methyl-N-piperazinamide), dibutyl D/L-tartrate, di-tert-butyl D/L-tartrate, diisopropyl D/L-tartrate, dimethyl D/L-tartrate or diethyl D/L-tartrate.

16. The process according to claim 1, characterized in that the zirconium component used is zirconium(IV) acetylacetonate, zirconium(IV) butoxide, zirconium(IV) tert-butoxide, zirconium(IV) ethoxide, zirconium(IV) n-propoxide, zirconium(IV) isopropoxide, or zirconium(IV) isopropoxide/isopropanol complex and that the process is carried out in the presence of a mixture of enantiomers of D/L-tartaric acid bis-(N,N-diallylamide), D/L-tartaric acid bis-(N,N-dibenzylamide), D/L-tartaric acid bis-(N,N-diisopropylamide), D/L-tartaric acid bis-(N,N-dimethylamide), D/L-tartaric acid bis-(N-pyrrolidinamide), D/L-tartaric acid bis-(N-piperidinamide), D/L-tartaric acid bis-(N-morpholinamide), D/L-tartaric acid bis-(N-cycloheptylamide), D/L-tartaric acid bis-(N-4-methyl-N-piperazinamide), dibutyl D/L-tartrate, di-tert-butyl D/L-tartrate, diisopropyl D/L-tartrate, dimethyl D/L-tartrate or diethyl D/L-tartrate and in the presence of an organic base.

17. The process according to claim 1, characterized in that the process is carried out in the presence of a mixture of enantiomers of D/L-tartaric acid bis-(N,N-dimethylamide), D/L-tartaric acid bis-(N-pyrrolidinamide) or D/L-tartaric acid bis-(N-morpholinamide) and in the presence of an organic base.

18. The process according to claim 1, characterized in that a mixture of enantiomers of 5-methoxy-2-[(4-methoxy-3,5-dimethyl-2-pyridinyl)methylsulphinyl]-1H-benzimidazole, 5-difluoromethoxy-2-[(3,4-dimethoxy-2-pyridinyl)methyl-sulphinyl]-1H-benzimidazole, 2-[3-methyl-4-(2,2,2-trifluoroethoxy)-2-pyridinyl)methylsulphinyl]-1H-benzimidazole, 2-{[4-[3-methoxypropoxy)-3-methylpyridin-2-yl]methylsulphinyl}-1H-benzimidazole or 5-methoxy-2-((4-methoxy-3,5-dimethyl-2-pyridylmethyl)sulphinyl/}-1H-imidazo(4,5-b)pyridine is prepared by the process.

19. The process according to claim 1, characterized in that the process is carried out in the presence of a mixture of enantiomers of D/L-tartaric acid bis-(N,N-dimethylamide), D/L-tartaric acid bis-(N-pyrrolidinamide) or D/L-tartaric acid bis-(N-morpholinamide) and that the process product prepared is of a mixture of enantiomers of pantoprazole.

20. The process according to claim 1, characterized in that the zirconium component used is zirconium(IV) n-propoxide, zirconium(IV) isopropoxide or zirconium(IV) isopropoxide/isopropanol complex, that the process is carried out in the presence of a mixture of enantiomers of D/L-tartaric acid bis-(N,N-dimethylamide), D/L-tartaric acid bis-(N-pyrrolidinamide) or D/L-tartaric acid bis-(N-morpholinamide) that the oxidation is carried out using cumene hydroperoxide and that the process product prepared is a mixture of enantiomers of pantoprazole.

21. The process according to claim 1, characterized in that the zirconium component used is zirconium(IV) n-propoxide, zirconium(IV) isopropoxide or zirconium(IV) isopropoxide/isopropanol complex, that the process is carried out in the presence of a mixture of enantiomers of D/L-tartaric acid bis-(N,N-dimethylamide), D/L-tartaric acid bis-(N-pyrrolidinamide) or D/L-tartaric acid bis-(N-morpholinamide) that the oxidation is carried out using cumene hydroperoxide in the presence of a tertiary amine and that the process product prepared is pantoprazole.

22. The process according to claim 1, characterized in that the zirconium component used is zirconium(IV) n-propoxide or zirconium(IV) isopropoxide complex, that the process is carried out in the presence of a racemic mixture of enantiomers of D/L-tartaric acid bis-(N-pyrrolidinamide), that the oxidation is carried out using cumene hydroperoxide in the presence of a tertiary amine and that the process product prepared is a racemic mixture of enantiomers of pantoprazole.

23. A mixture of enantiomers of 5-methoxy-2-[(4-methoxy-3,5-dimethyl-2-pyridinyl)methylsulphinyl]-1H-benzimidazole, 5-difluoromethoxy-2-[(3,4-dimethoxy-2-pyridinyl)methylsulphinyl]-1H-benzimidazole, 2-[3-methyl-4-(2,2,2-trifluoroethoxy)-2-pyridinyl)methylsulphinyl]-1H-benzimidazole, 2-{[4-(3-methoxypropoxy)-3-methylpyridin-2-yl]methylsulphinyl}-1H-benzimidazole or 5-methoxy-2-((4-methoxy-3,5-dimethyl-2-pyridylmethyl)sulphinyl)-1H-imidazo[4,5-b]pyridine prepared by the process according to claim 1.
